(19) United States
(12) Patent Application Publication
(10) Pub. No.: US 2022/0142715 A1
(43) Pub. Date: May 12, 2022

(54) ULTRASONIC DETECTION OF CATHETER LOCATION AND VEIN DEFORMATION DETECTION BY ABLATION CATHETER

(71) Applicant: Biosense Webster (Israel) Ltd., Yokneam (IL)

(72) Inventors: Morris Ziv-Ari, Atlit (IL); Eid Adawi, Tur'an (IL)

(73) Assignee: Biosense Webster (Israel) Ltd., Yokneam (IL)

(21) Appl. No.: 17/521,135

(22) Filed: Nov. 8, 2021

Related U.S. Application Data
(60) Provisional application No. 63/112,010, filed on Nov. 10, 2020.

(51) Int. Cl. A61B 34/20; A61B 8/08; A61B 18/14

(52) U.S. Cl. CPC A61B 34/20; A61B 8/0841; A61B 8/085; A61B 2018/00404; A61B 2034/2063; A61B 2034/2065; A61B 2034/2051; A61B 18/1492

(57) ABSTRACT

A system, device and method of using an ultrasound sensor with a balloon catheter are disclosed. The system, device and method include collecting an ultrasound reading from the ultrasound sensor, comparing a vein diameter to the collected ultrasound reading, and calculating an inflation index based on a generated map and the respective positions of the ACL electrodes. If the vein diameter is the same as the ultrasound reading, the balloon catheter is too far into the vein and needs to be retracted. The ultrasound reading may be compared to the inflation index of the balloon and/or to the vein diameter. The system, device and method include obtaining a baseline characterization of the vein (ostium), obtaining a current characterization of the vein (ostium) after a catheter is in position for a PV ablation, comparing the respective characterizations, and determining a location of the catheter based on the comparison.

### CROSS REFERENCE TO RELATED APPLICATIONS

This application claims the benefit of U.S. Provisional Application No. 63/112,010, filed Nov. 10, 2020, which is incorporated by reference as if fully set forth.

### FIELD OF INVENTION

The present invention relates to catheter location as a benefit during heart ablation, and more specifically, as an ultrasonic detection system and method for detecting a catheter location.

### BACKGROUND

Atrial fibrillation is an abnormal heart rhythm that originates in the top chambers of the heart (atria). In patients with atrial fibrillation, the SA node does not direct the heart's electrical rhythm. Instead, one or more different impulses may rapidly fire at once. This may cause a fast, chaotic or other abnormal rhythm in the atria. As a result, the atria may not contract and/or effectively squeeze blood into the ventricle. Atrial fibrillation may begin in the pulmonary veins or at the point where the pulmonary veins attach to the left atrium. There are four major pulmonary veins. All, or any, of the veins may trigger atrial fibrillation.

Atrial fibrillation may be corrected by ablating a portion of one or more pulmonary veins that are or may be causing the atrial fibrillation in order to isolate the electrical signals from the one or more pulmonary veins. Pulmonary vein ablation (also referred to as pulmonary vein isolation (PVI)) is a treatment for atrial fibrillation. A PVI treatment may be carried out using a balloon pump catheter that includes multiple electrodes along the surface of the balloon pump in a configuration to allow the multiple electrodes to ablate along a circumference of the pulmonary vein.

However, balloon pumps often are inserted too far into a pulmonary vein. An ablation carried out too far into a pulmonary vein can cause pulmonary vein stenosis (PVS) which is a serious condition in which there is a blockage in the blood vessels affecting the flow of blood from the lungs to the heart. This blockage is caused by an abnormal thickening of the walls of the pulmonary vein which result from ablating too far into the pulmonary vein.

### SUMMARY

A system, device and method of using an ultrasound sensor with a balloon catheter are disclosed. The system, device and method include collecting an ultrasound reading from the ultrasound sensor, comparing a vein diameter to the collected ultrasound reading, and calculating an inflation index based on a generated map and the respective positions of the ACL electrodes. If the vein diameter is the same as the ultrasound reading, the balloon catheter is too far into the vein and needs to be retracted. The ultrasound reading may be compared to the inflation index of the balloon and/or to the vein diameter.

A system, device and method of vein deformation detection by ablation catheter are also disclosed. The system, device and method include obtaining a baseline characterization of the vein (including ostium), obtaining a current characterization of the vein (including ostium) after a catheter is approximately in position for a PV ablation, comparing the baseline characterization to the current characterization, and determining a location of the catheter based on the comparison. The baseline characterization comprises a visual representation, such as an image captured or imaged by an imaging device, such as a camera, an ultrasound transducer, MRI device, etc., that collects the visual representation during baseline collection. The baseline characterization comprises a force detector that captures information, such as a rendering via electromagnetic sensor-based mapping-based rendering, for example, during a baseline collection including a pressure indicator, to provide the amount of pressure on an ostium during baseline collection.

### BRIEF DESCRIPTION OF THE DRAWINGS

A more detailed understanding may be had from the following description, given by way of example in conjunction with the accompanying drawings, wherein like reference numerals in the figures indicate like elements, and wherein:

FIG. 1 illustrates a depiction of an ultrasound sensor in a balloon catheter with the balloon catheter located inside the vein;

FIG. 2 illustrates a method of using the ultrasound sensor located inside the balloon catheter;

FIG. 3 illustrates a depiction of an ultrasound sensor in a balloon catheter with the balloon catheter located outside the vein;

FIG. 4 illustrates a baseline characterization resulting from the use of an imaging device in evaluating vein deformation detection by an ablation catheter;

FIG. 5 illustrates an exemplary characterization resulting from the use of an imaging device in evaluating vein deformation detection by an ablation catheter; and

FIG. 6 illustrates a method of vein deformation detection by ablation catheter.

### DETAILED DESCRIPTION

In order to achieve the goal of performing a PVI procedure where ablation occurs at the ostia (opening) of a pulmonary vein (PV) the following system and method are provided.

A system, device and method of using an ultrasound sensor with a balloon catheter are disclosed. The system, device and method include collecting an ultrasound reading from the ultrasound sensor, comparing a vein diameter to the collected ultrasound reading, and calculating an inflation index based on a generated map and the respective positions of the advanced current locations (ACL) electrodes. If the vein diameter is the same as the ultrasound reading, the balloon catheter is too far into the vein and needs to be retracted. The ultrasound reading may be compared to the inflation index of the balloon and/or to the vein diameter.

A system, device and method of vein deformation detection by ablation catheter are also disclosed. The system, device and method include obtaining a baseline characterization of the vein (including ostium), obtaining a current characterization of the vein (including ostium) after a catheter is approximately in position for a PV ablation, comparing the baseline characterization to the current characterization, and determining a location of the catheter based on the comparison. The baseline characterization comprises a visual representation, such as an image captured or imaged by an imaging device, such as a camera, an ultrasound transducer, MRI device, etc., that collects the visual representation during baseline collection. The baseline characterization comprises a force detector that captures information, such as a rendering such as via electromagnetic sensor-based mapping-based rendering during a baseline collection including a pressure indicator, to provide the amount of pressure on an ostium at during a baseline collection.

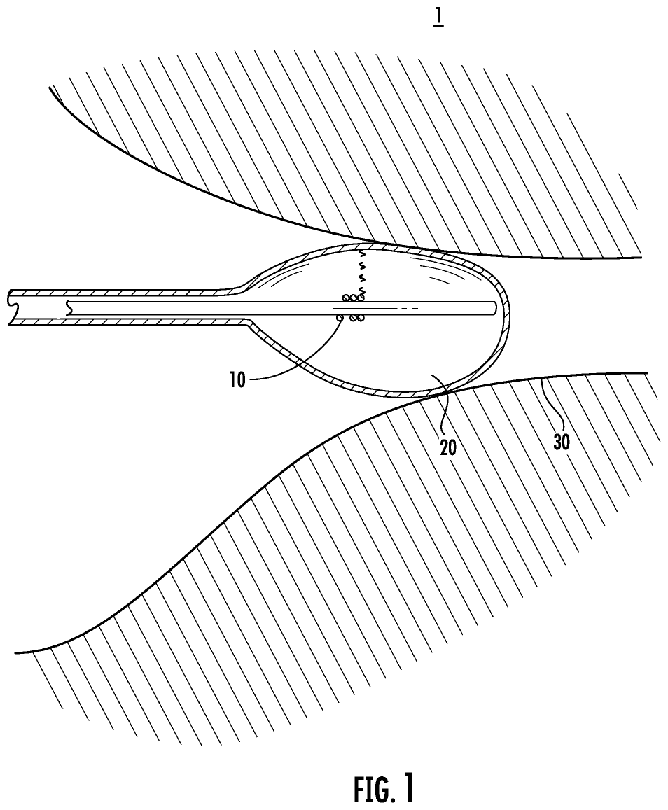

FIG. 1 illustrates a depiction 1 of an ultrasound sensor 10 in a balloon catheter 20 with the balloon catheter 20 located inside the vein 30. This ultrasound sensor 10 is depicted as being inside of the balloon catheter 20, although such a location is only provided as an exemplary location. Other locations for the ultrasound sensor 10 include outside of the balloon catheter 20.

In the exemplary depiction of FIG. 1 where the ultrasound sensor 10 is located inside the balloon catheter 20, the balloon catheter 20 may be transparent, at least at the ultrasound frequencies, to allow ultrasound data to be read as the signals pass through the transparent wall of the balloon catheter 20.

In an exemplary configuration, the ultrasound sensor 10 may be located in the balloon catheter 20 at a proximal end of the balloon catheter 20, such as closer to the sheath, for example. In this configuration, any ultrasound results that are received indicating a mass less than the radius of the balloon catheter 20 may be ignored as these results may be the ultrasound signal bouncing off the catheter 20 wall instead of the vein 30 or ostium as desired.

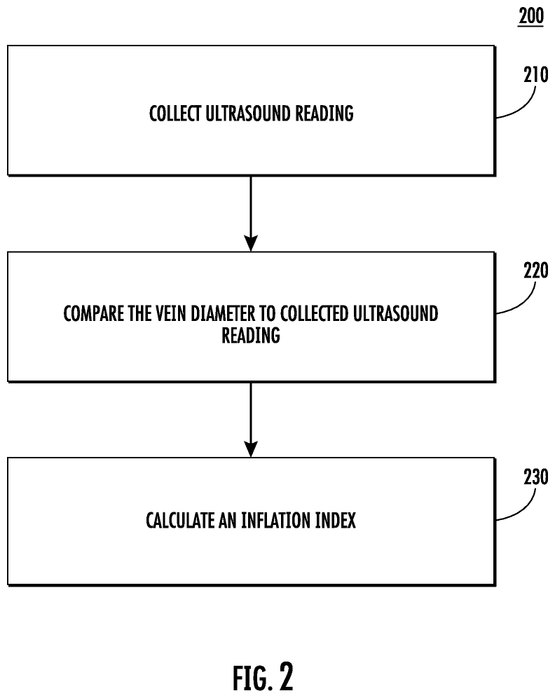

As shown in FIG. 1, the ultrasound sensor 10 from the inside of the balloon catheter 20 indicates that the catheter is located inside the vein 30. Such an indication is provided by employing the method of FIG. 2 described below in comparing the ultrasound to a catheter inflation index and/or by comparing ultrasound results to the vein diameter.

FIG. 2 illustrates a method 200 of using the ultrasound sensor (10 in FIG. 1) located inside the balloon catheter (20 in FIG. 1). The method 200 includes collecting an ultrasound reading from the ultrasound sensor at step 210. The method 200 includes comparing the vein diameter (either known, measured, or approximated) to the collected ultrasound reading at step 220. If the vein diameter is the same, or relatively so, as the ultrasound reading, this indicates that the balloon catheter is too far into the vein and needs to be retracted. The comparison may be made by comparing ultrasound reading to the vein diameter. Alternatively, or additionally, the comparison may be made such that the ultrasound reading may be compared to an inflation index of the balloon. The method 200 may include calculating an inflation index based on a generated map and the respective positions of the ACL electrodes at step 230. The inflation index provides the diameter of the balloon. If this is the same diameter as the ultrasound, then the balloon catheter is too far into the vein.

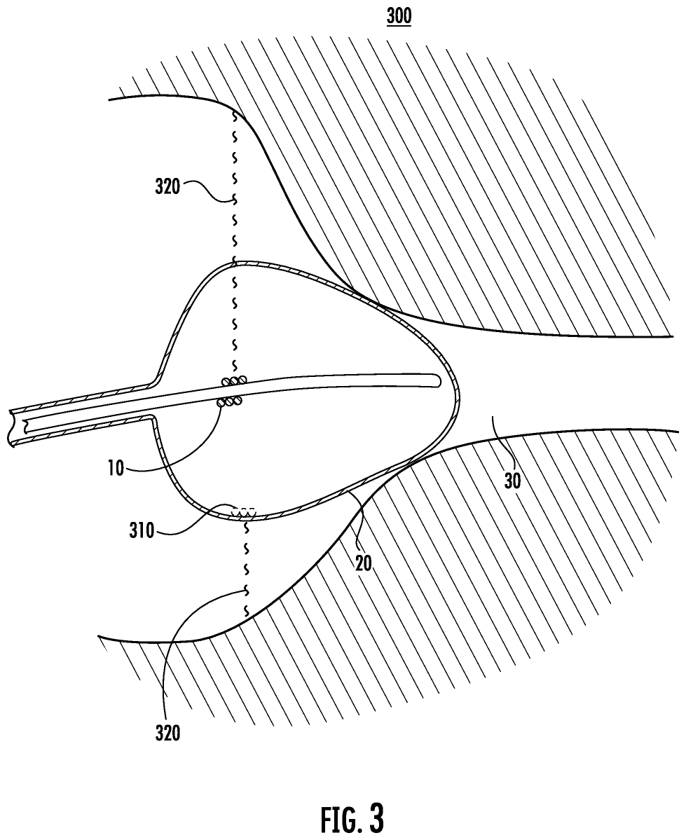

FIG. 3 illustrates a depiction 300 of an ultrasound sensor 10 in a balloon catheter 20 with the balloon catheter 20 located outside the vein 30. There are two depicted ultrasound sensors in FIG. 3. A first option, similar to that depicted in FIG. 1, includes the ultrasound sensor 10 inside the balloon catheter 20. A second option of the ultrasound sensor illustrates an ultrasound sensor 310 located on the outside of the balloon catheter 20. While these two locations, inside and outside, are provided as options, both configurations may be utilized simultaneously.

As shown in FIG. 3, the ultrasound from the inside/outside of the balloon catheter 20 indicates that the catheter is located outside (evidenced by the width shown as 320) of the vein 30. Such an indication is provided by the method of FIG. 2 in comparing the ultrasound to a catheter inflation index and/or by comparing ultrasound results to the vein diameter.

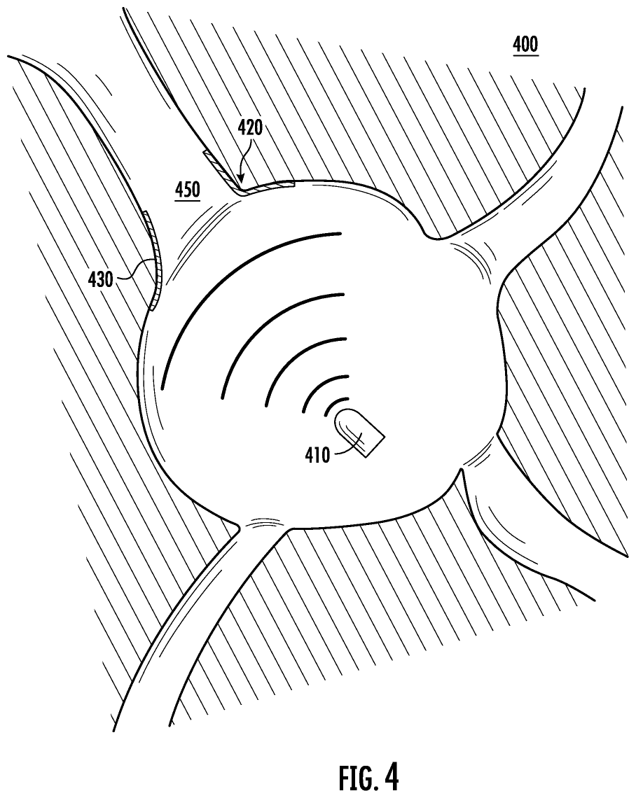

The present configuration using ultrasound catheter with the balloon may also provide for vein deformation detection by an ablation catheter. Two configurations for monitoring the vein deformation may be utilized. In a first embodiment, an imaging device is utilized to compare a baseline image to a current characterization image. The first embodiment is illustrated in FIG. 4. Specifically, FIG. 4 illustrates a baseline characterization 400 resulting from the use of an imaging device 410 in evaluating vein deformation detection by an ablation catheter.

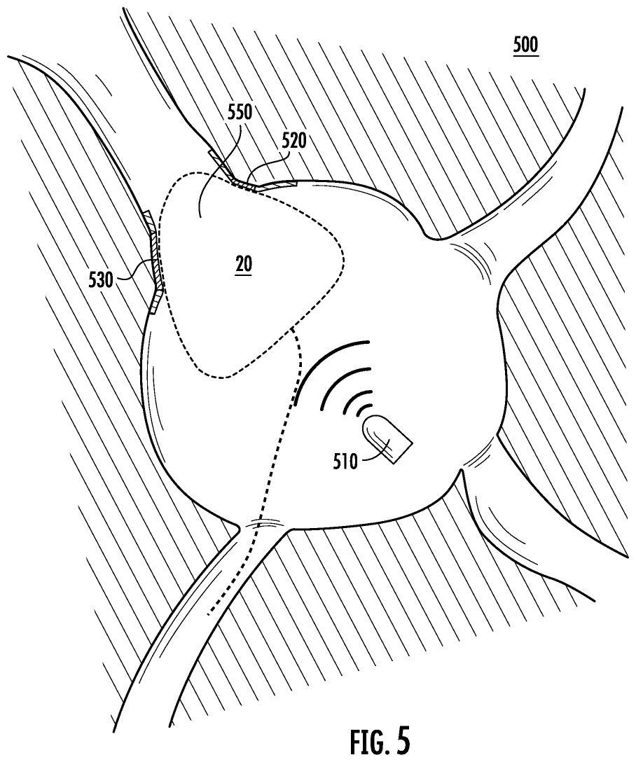

In a second embodiment, force information is utilized to compare a baseline of the force information to a current characterization of the force information. The second embodiment is illustrated in FIG. 5. Specifically, FIG. 5 illustrates a current characterization 500 resulting from the use of an imaging device 510 in evaluating vein deformation detection by an ablation catheter.

Both embodiments may be used individually, or in combination, to perform a PVI procedure where ablation is carried out at the ostia (opening) of a pulmonary vein (PV) as desired. Using the baseline image illustrated in FIG. 4 and the current image illustrated in FIG. 5, the current visual property of the ostia to a baseline visual property of the ostia is compared to determine if the catheter is positioned on the ostia, e.g., applying pressure on the walls that create the ostia.

Specifically, in FIG. 4, the visual property of the ostia 450 is evidenced by points 420, 430. In FIG. 5, with the balloon catheter 20 inserted in place, the ostia 550 is evidenced by points 520, 530. A comparison of points 420, 430 to points 520, 530 may be used to determine if the catheter 20 is positioned on the ostia 450, 550. The comparison may be performed to determine if there is pressure from the balloon catheter 20 on the ostia 450, 550 walls.

In combination with FIGS. 4 and 5, FIG. 6 illustrates a method 600 of vein deformation detection (observed between points 420, 430 and points 520, 530 of FIGS. 4 and 5) by ablation catheter 20. The method 600 includes obtaining a baseline characterization of the vein (including ostium) at step 610, illustrated specifically in FIG. 4 (points 420, 430) for the imaging embodiment. As shown in FIG. 4, this may include a visual representation. For example, the visual representation or image may be captured or imaged, e.g., via an imaging device, such as a camera, an ultrasound transducer, MRI device, etc., that collects the visual representation during baseline collection. Alternatively, or additionally, a force detector may be used, e.g., via a rendering such as via electromagnetic sensor-based mapping-based rendering during a baseline collection. This may include a pressure indicator that provides the amount of pressure on an ostium at during a baseline collection.

The method 600 includes obtaining a current characterization of the vein (including ostium) after a catheter is approximately in position for a PV ablation at step 620. This configuration is illustrated in FIG. 5 (points 520, 530). This current characterization may utilize the same visual representation as step 610, e.g., via an imaging device including an ultrasound catheter that collects the visual representation during a current collection, or force detector, e.g., via a rendering via electromagnetic sensor-based mapping-based rendering during a current collection including a pressure indicator that provides the amount of pressure on an ostium at a baseline position during a current collection, utilized during baseline characterization.

The method 600 may include comparing the baseline characterization to the current characterization at step 630. The comparison may be performed visually or automatically, by comparing the images using an image comparison software, for example. The method 600 may include determining a location of the catheter based on the comparison at step 640. This determination may be calculated automatically such as by software, for example.

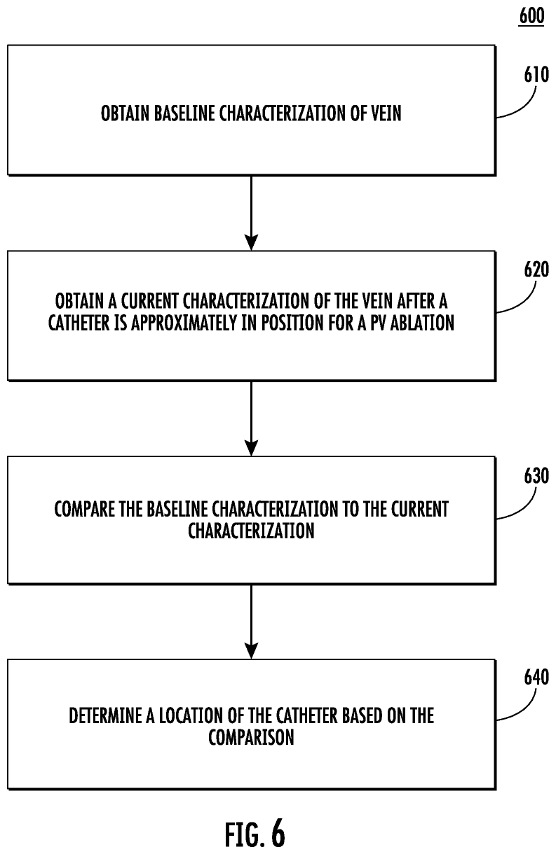

Using the method 600 of FIG. 6, a determination as to the contact of the catheter with the ostium, whether the contact of the catheter with the ostium occurs at an appropriate position allowing ablation to be conducted properly, and whether the catheter is applying too much pressure on the ostium, indicating that ablation may be too deep into the PV, and illustrating that the catheter should be retracted.

The present embodiments include an alert that is provided if too much pressure is applied on the ostium, an automatic application to track the change in vein and using a threshold for the alert.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random-access memory (RAM), a register, cache memory, semiconductor memory devices, magnetic media such as internal hard disks and removable disks, magneto-optical media, and optical media such as CD-ROM disks, and digital versatile disks (DVDs).

1. A method of using an ultrasound sensor with a balloon catheter comprising:
collecting an ultrasound reading from the ultrasound sensor, the ultrasound reading including the balloon catheter and a vein;
determining a diameter of the vein;
comparing the vein diameter to the collected ultrasound reading;
generating a map of the vein and the balloon catheter; and
calculating an inflation index based on the generated map and the respective positions of the advanced current location (ACL) electrodes.

2. The method of claim 1, wherein if the vein diameter is the same as the ultrasound reading, the balloon catheter is too far into the vein and needs to be retracted.

3. The method of claim 2, wherein the same includes readings that are substantially similar.

4. The method of claim 1, further comprising comparing the ultrasound reading to the inflation index of the balloon.

5. The method of claim 1, further comprising comparing the ultrasound reading to the vein diameter.

6. The method of claim 1, wherein the inflation index provides the diameter of the balloon.

7. A method of vein deformation detection by ablation catheter, the method comprising:
obtaining a baseline characterization of the vein;
obtaining a current characterization of the vein after a catheter is approximately in position for a PV ablation;
comparing the baseline characterization to the current characterization; and
determining a location of the catheter based on the comparison.

8. The method of claim 7 wherein the vein includes the ostium.

9. The method of claim 7, wherein the baseline characterization comprises a visual representation.

10. The method of claim 9, wherein the visual representation comprises an image captured or taken by an imaging device, such as a camera, an ultrasound transducer, MRI device, etc., that collects the visual representation during a baseline collection.

11. The method of claim 7, wherein the baseline characterization comprises a force detector information.

12. The method of claim 11, wherein the force detector comprises a rendering such as via electromagnetic sensor-based mapping-based rendering during a baseline collection.

13. The method of claim 12, wherein the force detector comprises a pressure indicator that provides the amount of pressure on an ostium at during a baseline collection

14. The method of claim 7, wherein the comparing is performed visually or automatically, by comparing the images using an image comparison software.

15. The method of claim 15, wherein the determination is calculated automatically by software.

16. A system for detecting vein deformation by an ablation catheter, the system comprising:
an ablation catheter;
an ultrasound reading of the vein and the ablation catheter when the ablation catheter is approximately in position for a PV ablation; and
a processor communicatively coupled to a data input/output,
the processor obtaining a baseline characterization of the vein, obtaining a current characterization of the vein after a catheter is approximately in position for a PV ablation, comparing the baseline characterization to the current characterization, and determining a location of the catheter based on the comparison.

17. The system of claim 16, wherein the baseline characterization comprises a visual representation.

18. The system of claim 17, wherein the visual representation comprises an image captured or taken by an imaging device, such as a camera, an ultrasound transducer, MRI device, etc., that collects the visual representation during a baseline collection.

19. The system of claim 16, further comprising a force detector that measures force detector information for inclusion in the baseline characterization.

20. The system of claim 19, wherein the force detector comprises a rendering such as via electromagnetic sensor-based mapping-based rendering during a baseline collection.

21. The system of claim 20, wherein the force detector comprises a pressure indicator that provides the amount of pressure on an ostium at during a baseline collection.
